(19) United States
(12) Patent Application Publication
(10) Pub. No.: US 2022/0184157 A1
(43) Pub. Date: Jun. 16, 2022

(54) ONCOLYTIC VIRUS AND FOCUSED ULTRASOUND FOR NON-INVASIVE CNS FOCAL GENE DELIVERY

(71) Applicants: CORNELL UNIVERSITY, Ithaca, NY (US); THE ROCKEFELLER UNIVERSITY, New York, NY (US); The UAB Research Foundation, Birmingham, AL (US)

(72) Inventors: Michael G. Kaplitt et al., New York, NY (US); Michaela Stsvarache, New York, NY (US); Jsmes Markert, Birmingham, AL (US)

(21) Appl. No.: 17/442,732

(22) PCT Filed: Mar. 27, 2020
(86) PCT No.: PCT/US2020/025463

§ 371 Date: Sep. 24, 2021

Related U.S. Application Data
(60) Provisional application No. 62/824,685, filed on Mar. 27, 2019.

(51) Int. Cl. A61K 35/763; A61N 7/00; C12N 7/00; A61P 35/00

(52) U.S. Cl. CPC A61K 35/763; A61N 7/00; A61N 2007/0039; A61P 35/00; C12N 7/00

(57) ABSTRACT

Methods of employing oncolytic viruses and focused ultrasound, e.g., for focal delivery to the mammalian brain, are provided.

### CROSS-REFERENCE TO RELATED APPLICATIONS

This application claims the benefit of the filing date of U.S. application No. 62/824,685, filed on Mar. 27, 2019, the disclosure of which is incorporated by reference herein.

### BACKGROUND

Gene therapy has proven to be safe and effective for a variety of neurological and oncological diseases in both preclinical studies and in human trials. One limitation, however, has been the continued need for direct injection of gene therapy agents into the brain. This creates risks of invasive surgery, such as hemorrhage. It also can be very difficult to tailor the delivery of agents to optimally cover desired brain targets with direct infusion, since the direction and coverage of fluid flow is limited by the need to deliver from a single point at the end of an infusion catheter. For tumors of the central nervous system, this is particularly problematic since tumor recurrence following surgery and adjunctive therapy is usually due to cells which have invaded the otherwise normal brain tissue surrounding the visible mass and direct infusion of gene therapy agents into the surrounding normal brain tissue to attack invading tumor cells can be difficult and highly inefficient due to altered tissue resistance and flow dynamics in the presence of a resection cavity. Systemic delivery of certain gene therapy agents can lead to transduction in the brain, due to the ability to cross the blood-brain barrier (BBB). However, these are not only generally inefficient, but they also cannot be targeted to particular areas of the brain. For many applications, for example in neurodegenerative and psychiatric diseases, global delivery of genes throughout the brain and spinal cord would not be optimal since they could cause untoward effects from genetically modifying regions of the brain or spinal cord which might have effects contrary to the therapeutic goals of gene delivery to the desired target. Systemic delivery of gene therapy agents may also result in transduction of peripheral organs, which can lead to adverse effects due to unnecessary and undesired gene expression in peripheral organs.

### SUMMARY

The disclosure provides for a method of delivering an oncolytic virus to one or more regions of a brain or spinal cord of a mammal having metastatic tumor cells within nervous system tissue. In one embodiment, the method includes administering a first amount of an oncolytic virus to the mammal and applying to one or more regions of the brain or spinal cord of the mammal suspected of having metastatic tumor cells focused ultrasound in an amount that provides for delivery of the oncolytic virus to the tumor cells. In one embodiment, the method includes administering an amount of a population of microbubbles and a first amount of an oncolytic virus to the mammal and applying to one or more regions of the brain or spinal cord of the mammal suspected of having metastatic tumor cells focused ultrasound in an amount that provides for delivery of the oncolytic virus to the tumor cells. In one embodiment, the oncolytic virus comprises a herpes simplex virus, poliovirus, reovirus or adenovirus. In one embodiment, the method includes subsequently administering a second amount of an oncolytic virus. In one embodiment, the oncolytic virus is delivered to brain or spinal cord tissue surrounding a resection cavity. In one embodiment, the oncolytic virus expresses a transgene capable of stimulating the immune system to attack tumor cells.

Transient focal opening of the blood brain barrier (BBB) by MR-guided focused ultrasound (MRgFUS) may allow for non-invasive CNS gene therapy to target precise brain regions. In one example, magnetic resonance (MR) guided focused ultrasound is used in combination with an oncolytic virus, to facilitate gene delivery to a particular region of the brain. In one example, magnetic resonance (MR) guided focused ultrasound is used in combination with microbubbles and an oncolytic virus, to facilitate gene delivery to a particular region of the brain. In one embodiment, microbubbles are injected intravenously immediately before or during the procedure to delivery ultrasound to one or more central nervous system target(s). In one embodiment, microbubbles are delivered simultaneously with the oncolytic virus.

In one embodiment focused ultrasound under the control of magnetic resonance imaging (MRI) is followed by administration, e.g., intravenous infusion, of an oncolytic virus. The method thus includes administering to the mammal an amount of an oncolytic virus and applying focused ultrasound to one or more regions of the central nervous system or the periphery of the mammal in an amount that allows the oncolytic virus to cross the blood brain barrier or enter tissue in the periphery. In one embodiment, the focused ultrasound is applied to the striatum, hippocampus or basal forebrain. In one embodiment, MR imaging is employed before and/or after the focused ultrasound. In one embodiment, frameless navigation is employed. In one embodiment, the focused ultrasound is applied after the the oncolytic virus is administered. In one embodiment, the focused ultrasound is applied concurrently with the administration of the oncolytic virus. In one embodiment, the oncolytic virus is directly injected. In one embodiment, the the oncolytic virus is systemically administered. In one embodiment, the oncolytic virus comprises an adenovirus or herpes simplex virus.

In one embodiment focused ultrasound under the control of magnetic resonance imaging (MRI) in combination with microbubbles (MB) formed of lipid-coated gas microspheres, followed by administration, e.g., intravenous infusion, of an oncolytic virus. The method thus includes administering to the mammal an amount of a plurality of microbubbles and an amount of an oncolytic virus and applying focused ultrasound to one or more regions of the central nervous system or the periphery of the mammal in an amount that allows the oncolytic virus to cross the blood brain barrier or enter tissue in the periphery. In one embodiment, the focused ultrasound is applied to the striatum, hippocampus or basal forebrain. In one embodiment, MR imaging is employed before and/or after the focused ultrasound. In one embodiment, frameless navigation is employed. In one embodiment,

the focused ultrasound is applied after the microbubbles and the oncolytic virus are administered. In one embodiment, the focused ultrasound is applied concurrently with the administration of the microbubbles and the oncolytic virus.

In one embodiment, the microbubbles and the oncolytic virus are directly injected. In one embodiment, the microbubbles and the oncolytic virus are systemically administered. In one embodiment, the oncolytic virus comprises an adenovirus or herpes simplex virus.

### BRIEF DESCRIPTION OF FIGURES

FIG. 1. Oncolytic HSV (oHSV) gene transfer into cerebral cortex 2 days following single focused ultrasound (FUS)-mediated blood brain barrier (BBB) disruption. Anti-DsRed antibody demonstrated expression of the transgene from the oHSV vector into the targeted cerebral cortex (top left) at 2 days following systemic viral delivery and FUS BBB disruption, with no expression in the contralateral cortex outside of the FUS field. NeuN staining shows no loss of neurons in the treated cortex compared with untreated cortex. GFAP staining shows minimal gliosis in the treated area compared with the untreated side. DAPI staining shows no evidence of neuronal death.

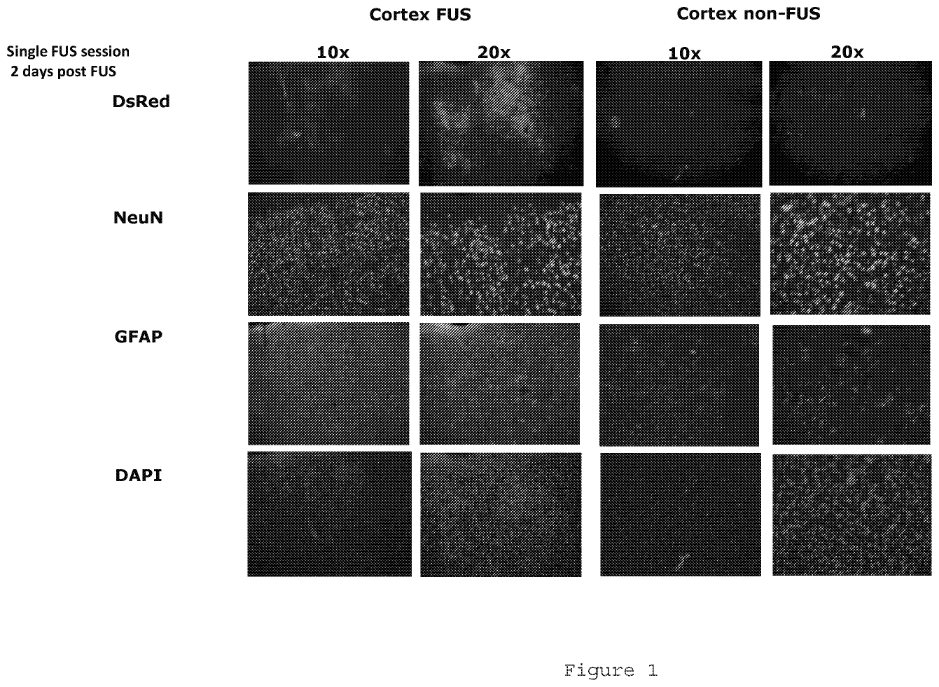

FIG. 2. 0HSV gene transfer into striatum 2 days following single focused ultrasound (FUS)-mediated blood brain barrier (BBB) disruption. Anti-DsRed antibody demonstrated expression of the transgene from the oHSV vector into the targeted striatum (top left) at 2 days following systemic viral delivery and FUS BBB disruption, with no expression in the contralateral striatum outside of the FUS field. NeuN staining shows no loss of neurons in the treated cortex compared with untreated cortex. GFAP staining shows minimal gliosis in the treated area compared with the untreated side. DAPI staining shows no evidence of neuronal death.

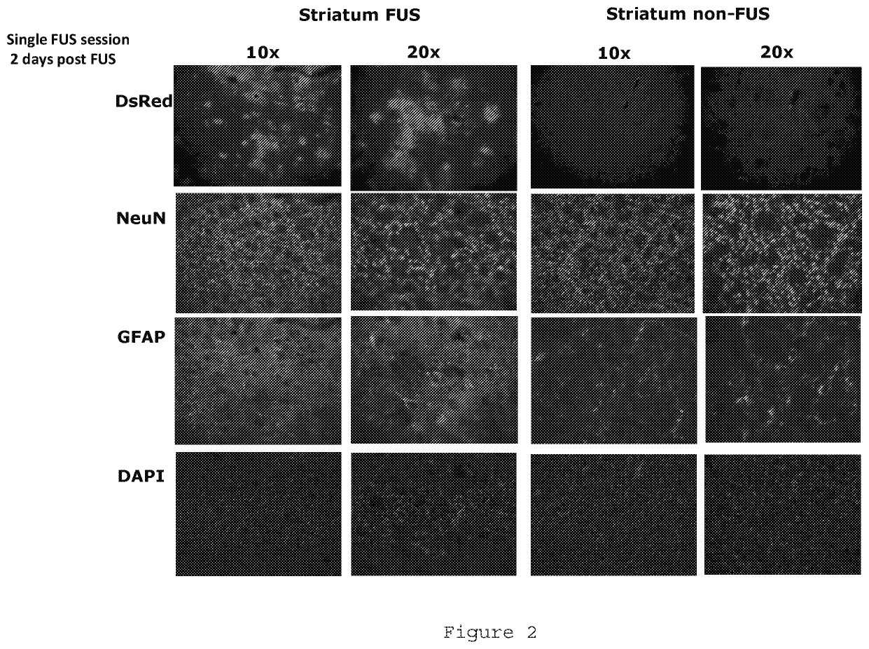

FIG. 3. Loss of gene expression at 8 days following oHSV delivery to cerebral cortex following MRgFUS BBB disruption. Little ongoing DsRed expression is observed in the treated cortex (top left) compared with the untreated side (top right) at 8 days following MRgFUS BBB disruption and systemic oHSV delivery compared with robust expression after 2 days. NeuN continues to show absence of neuronal loss on the treated compared with untreated sides, and GFAP shows slight gliosis on the treated compared with untreated side. DAPI staining shows no evidence of substantial cell death.

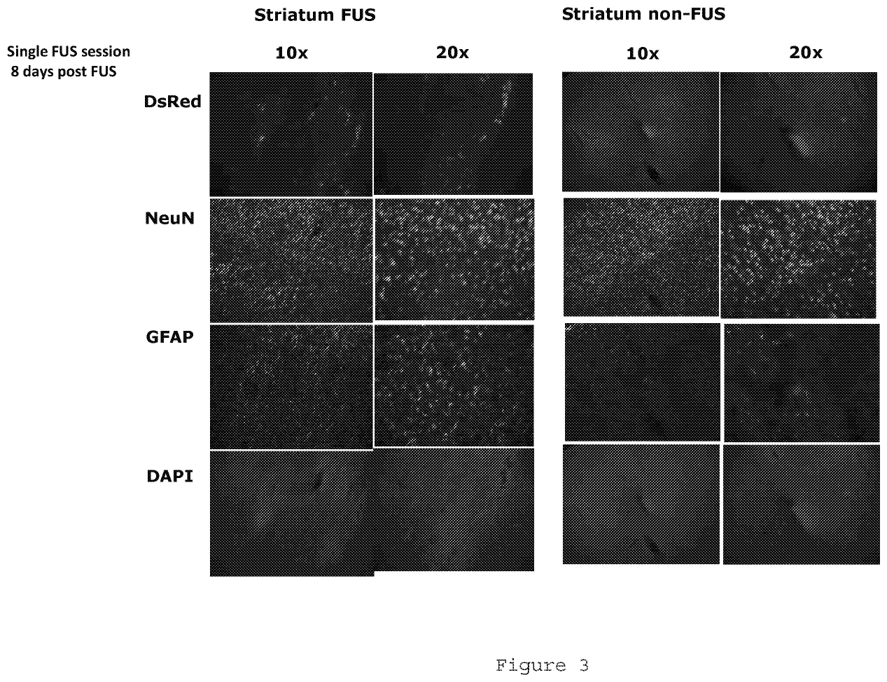

FIG. 4. Loss of gene expression at 8 days following oHSV delivery to striatum following MRgFUS BBB disruption. Little ongoing DsRed expression is observed in the treated striatum (top left) compared with the untreated side (top right) at 8 days following MRgFUS BBB disruption and systemic oHSV delivery compared with robust expression after 2 days. NeuN continues to show absence of neuronal loss on the treated compared with untreated sides, and GFAP shows no gliosis on the treated compared with untreated side. DAPI staining shows no evidence of substantial cell death.

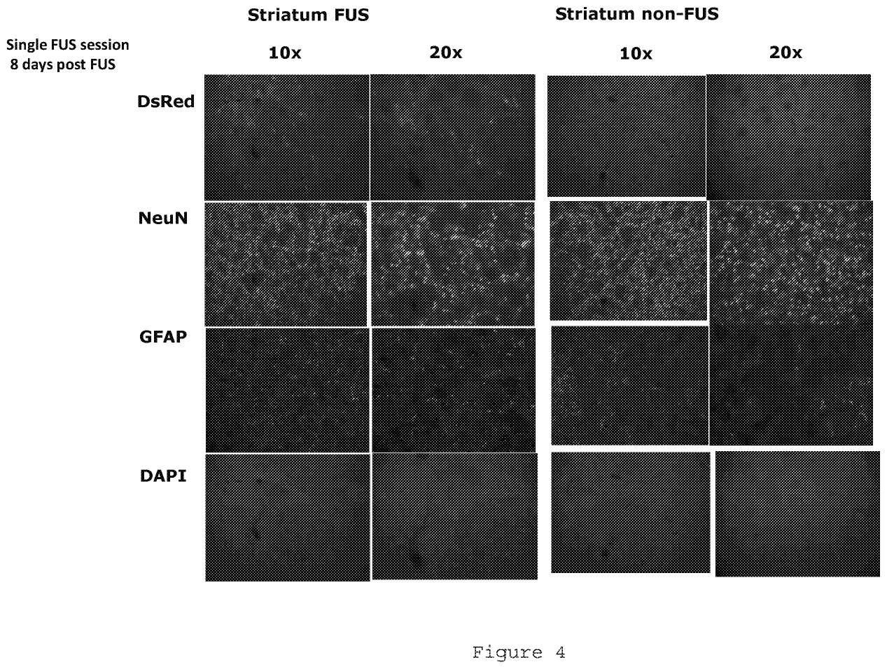

FIG. 5. Robust oHSV delivery and gene expression in cerebral cortex 2 days following repeated MRgFUS BBB disruption and repeated oHSV delivery. Animals were treated with oHSV systemically and subjected to MRgFUS BBB disruption in the cortex. 8 days later, after gene expression is lost, a second session of systemic oHSV followed by MRgFUS BBB disruption was performed targeting the same area. Top left shows robust DsRed expression 2 days after the second session of MRgFUS BBB disruption and oHSV delivery despite previous exposure to the same oHSV vector with a previous MRgFUS BBB disruption session, compared with no expression on the untreated hemisphere. Again there is no difference in NeuN or DAPI staining, indicating no substantial neuronal loss or cell death, and minimal gliosis.

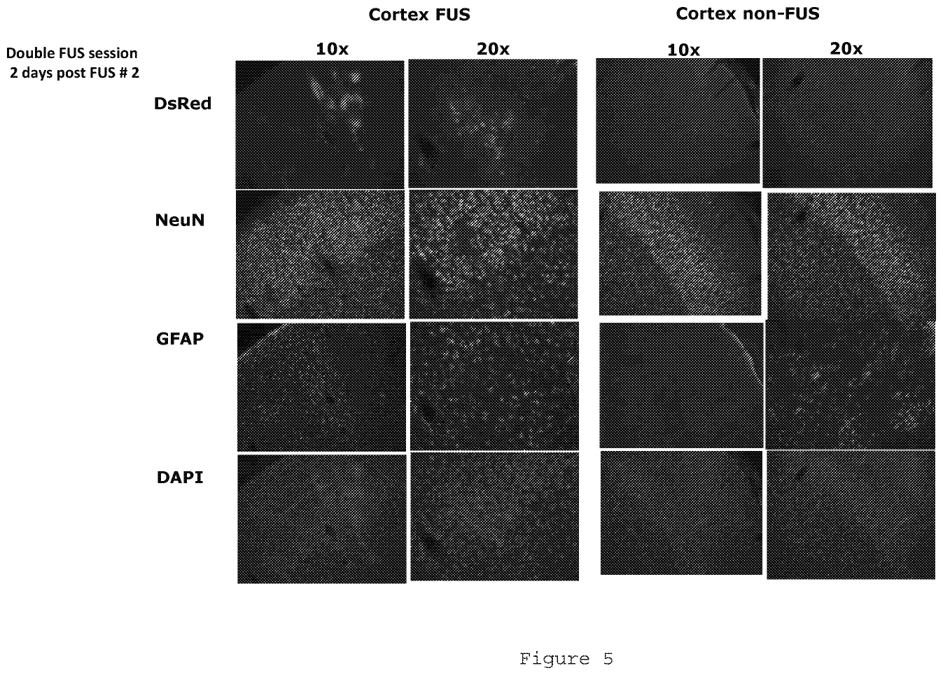

FIG. 6. Robust oHSV delivery and gene expression in striatum 2 days following repeated MRgFUS BBB disruption and repeated oHSV delivery. Animals were treated with oHSV systemically and subjected to MRgFUS BBB disruption in the striatum. 8 days later, after gene expression is lost, a second session of systemic oHSV followed by MRgFUS BBB disruption was performed targeting the same area. Top left shows robust DsRed expression 2 days after the second session of MRgFUS BBB disruption and oHSV delivery despite previous exposure to the same oHSV vector with a previous MRgFUS BBB disruption session, compared with no expression on the untreated hemisphere. Again there is no difference in NeuN or DAPI staining, indicating no substantial neuronal loss or cell death, and minimal gliosis.

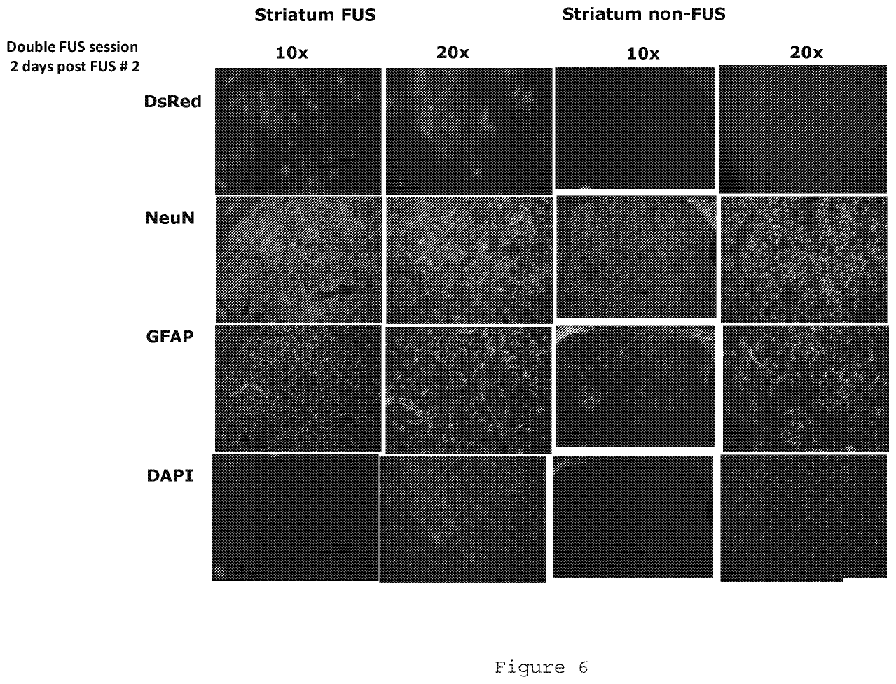

FIG. 7. MRgFUS BBB disruption for oHSV delivery to brain tissue surrounding a resection cavity. Top row: Pre-MRgFUS T1 MRI images of the rat brain. Middle row: Post-MRgFUS TI images with contrast show gadolinium extravasation into the brain in the vicinity of the resection cavity suggesting BBB disruption. Bottom row: SWAN image showing a hole in the cortex (red arrows) reflecting the location of the resection cavity.

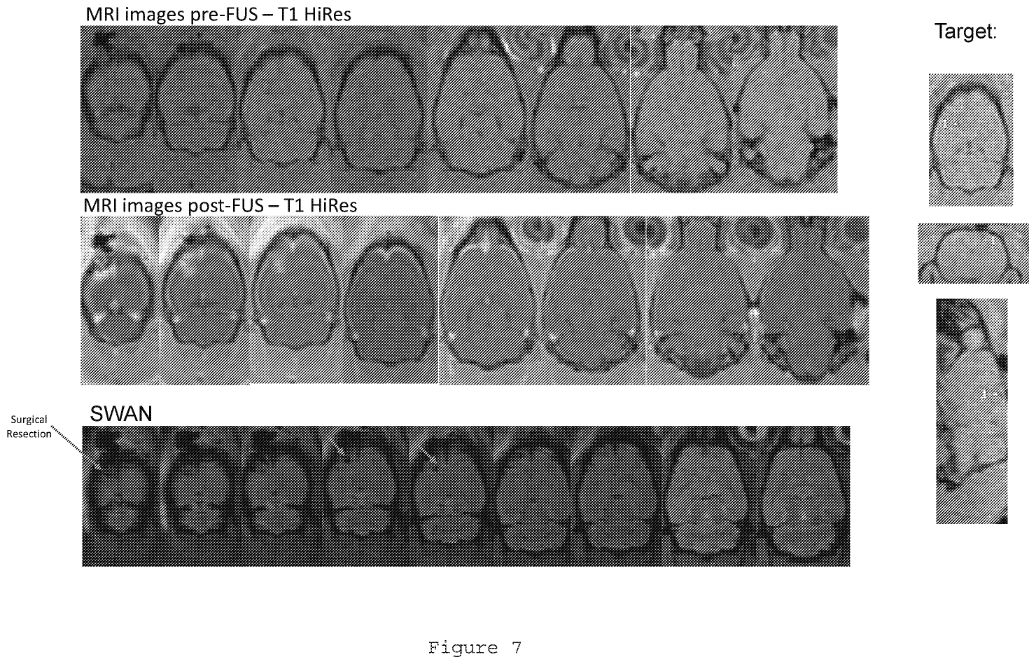

FIG. 8. Low magnification image of red fluorescent protein (RFP) expression reflecting oHSV delivery to brain tissue around resection cavity.

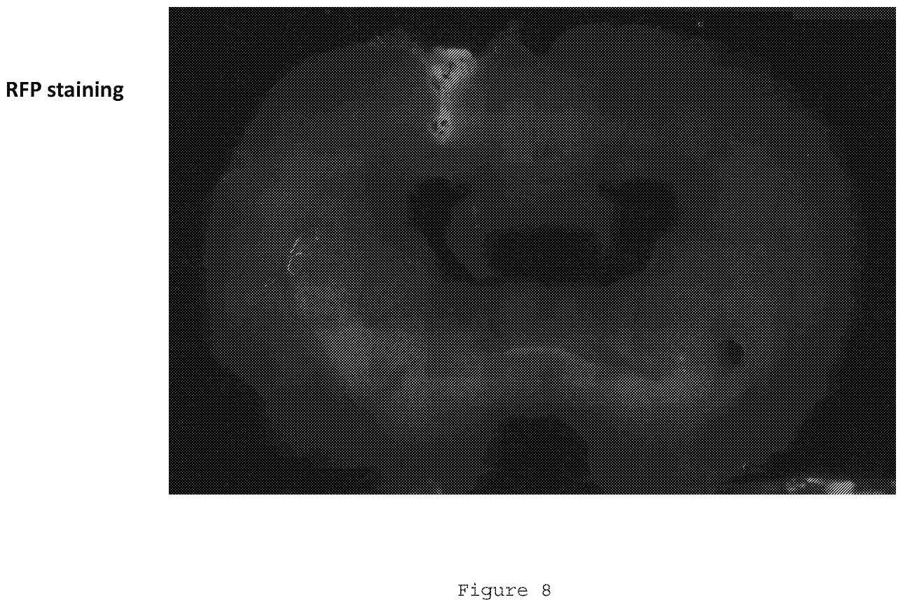

FIG. 9. 10× magnification of the section from FIG. 8 demonstrating oHSV-mediated gene expression preferentially in the brain tissue surrounding the resection cavity with no expression in the untreated side.

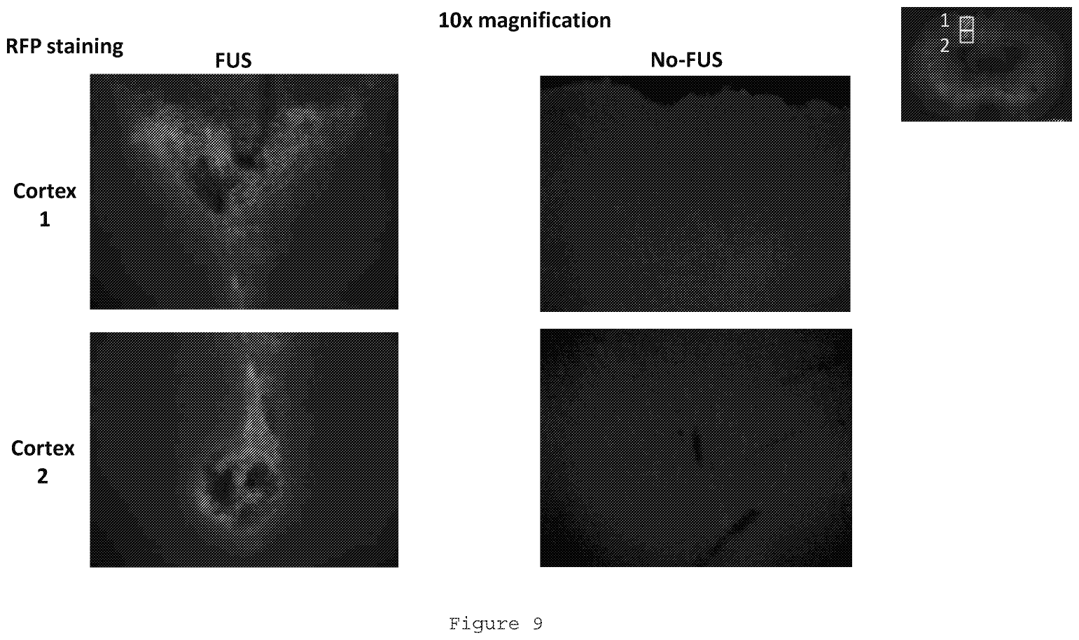

FIG. 10. MRgFUS blood brain barrier (BBB) disruption of normal brain surrounding a resection cavity. Brain tissue was resected using a suction technique. One month after resection, a resection cavity (red arrow) can be seen in all sequences. Top row: T1 MRI without contrast before focused ultrasound shows no enhancement. Second row: T1 MRI with gadolinium contrast before focused ultrasound shows no enhancement, indicating that the resection itself is not causing BBB breakdown and enhancement. Third row: T1 MRI with gadolinium contrast after focused ultrasound BBB disruption showing enhancement in the target tissue around the resection cavity, demonstrating effective BBB disruption in the brain tissue surrounding the resection cavity.

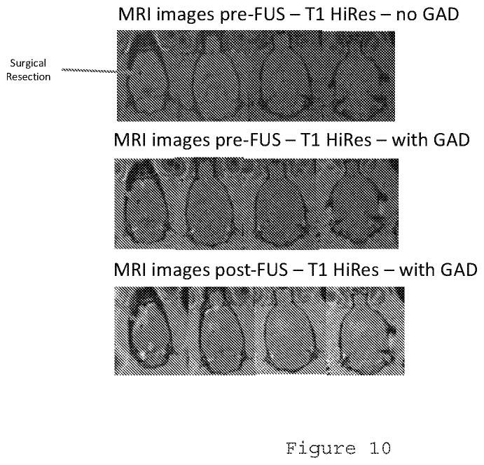

### DETAILED DESCRIPTION

Due to the presence of the blood-brain-barrier (BBB) and its highly selective permeability (Abbott et al., 2010; McCaffrey & Davis 2012), the only current means for efficient delivery of viral vectors to specific regions in the human brain has been through invasive direct injection. This not only carries the attendant risks of invasive surgery, but efficient distribution of gene therapy agents throughout a target area can be difficult to confirm with traditional infusion methods. Newer approaches have been tested, which permit monitoring of contrast spread during infusion as a surrogate for viral vector distribution utilizing specialized catheter systems with intraoperative magnetic resonance imaging (MRI) methodology (Fiandaca et al., 2009; Salegio et al., 2012).

In order to reduce surgical risks and avoid complexities of direct infusion, non-invasive approaches have been explored to permit intravenous delivery of viral vector into the brain. Use of an osmotic agent such as mannitol has long been known to transiently open the BBB, permitting delivery of a variety of agents to the brain, including viral vectors (Carty et al., 2010: Neuwelt et al., 1985; Neuwelt et al., 1979; Schuster et al., 2014). However, systemic administration of mannitol induces widespread opening of the BBB, precluding target specific gene expression, lective intra-arterial delivery of BBB disruption agents could provide more targeting gene delivery and cover larger brain areas, but variability in vascular supply of various important deep brain structures creates challenges for reproducible delivery between individuals (Foley et al., 2014).

An alternative to chemical delivery is mechanical disruption of the BBB. One approach is MR guided focused ultrasound (MRgFUS) (Hynynen et al., 2007; Hynynen et al., 2001; McDannold et al., 2005). This involves focused delivery of ultrasound to a target region, and high frequency MRgFUS has been used in human patients to create targeted brain lesions to treat essential tremor and pain (Elias et al., 2013; Elias et al., 2016; Jeanmonod et al., 2012). Use of MRgFUS at lower frequency, in combination with microbubble-mediated cavitation, has been shown to focally open the BBB to facilitate transfer of drugs (Treat et al., 2012), antibodies (Jordao et al., 2013), and nanoparticles (Nance et al., 2014) from the blood stream to the brain parenchyma. This has also been utilized for non-invasive gene delivery to the rodent brain (Alonso et al., 2013; Hsu et al., 2013; Huang et al., 2012; Thevenot et al., 2012; Wang et al., 2015). These reports have shown successful transfer of adeno-associated virus (AAV) vectors from the blood stream to the brain following MRgFUS-mediated BBB opening with variable efficiencies and with transgene expression evaluated for relatively short periods following delivery (Alonso et al., 2013; Hsu et al., 2013; Thevenot et al., 2012; Wang et al., 2015). Since the goal of gene delivery is long-term neuronal modification, long-term expression following MRgFUS-mediated gene delivery remains to be confirmed. This is particularly important since potential immune-mediated loss of gene expression or transduced cells, due to exposure of the brain to the immune system following BBB disruption, might not fully manifest until later time points, as has been observed in some gene transfer studies outside of the brain (Bell et al., 2011; Breous et al., 2011; Manno et al., 2006: Mingozzi et al., 2011; Wang et al., 2005). Furthermore, the potential for provoking inflammation in brain parenchyma following the application of focused ultrasound has only been examined up to 2 weeks following either MRgFUS BBB disruption alone (Jordao et al., 2013; Kovacs et al., 2017) or following delivery of AAV vectors (Thevenot et al., 2012; Wang et al., 2017). The potential consequences of long-term brain exposure to a potential immunogenic viral vector following BBB disruption remain unknown.

As described herein below, MRgFUS-mediated BBB disruption can lead to efficient delivery and wide distribution of oncolytic virus to the intended brain target.

### EXEMPLARY EMBODIMENTS

In one embodiment, the disclosure provides a method of delivering oncolytic virus to the brain or spinal cord. The method includes transiently disrupting the blood-brain barrier in a targeted brain region of a mammal using focused ultrasound and administering, e.g., systemic delivery of, an oncolytic virus. In one embodiment, the ultrasound field is targeted to a brain region using MRI guidance. In one embodiment, the method further comprises administering microbubbles, e.g., intravenously, in an amount to facilitate transient opening of the blood-brain barrier. In one embodiment, the oncolytic virus is delivered intravenously or intra-arterially. In one embodiment, the oncolytic virus comprises an adenovirus or herpes simplex virus. In one embodiment, the method does not employ the use of an osmotic agent.

Further provided is a method of delivering an oncolytic virus to the brain or spinal cord in order to target metastatic tumor cells within nervous system tissue. The method includes injection, e.g., systemic injection, of the oncolytic virus and transient disruption of the blood-brain using ultrasound to facilitate entry of the oncolytic virus into the brain. In one embodiment, the oncolytic virus comprises a herpes simplex virus, poliovirus, reovirus or adenovirus. In one embodiment,

the oncolytic virus is delivered in repeated sessions, e.g., each separated by at least one week. In one embodiment, the oncolytic virus is delivered to brain or spinal cord tissue surrounding a resection cavity. In one embodiment, the oncolytic virus expresses a transgene capable of stimulating the immune system to attack tumor cells. In one embodiment, the oncolytic virus is delivered in repeated sessions, e.g., each separated by more than one week.

The invention thus provides materials and methods useful for ultrasound-mediated non-invasive delivery of oncolytic viruses to the central nervous system. In one example, MR guided focused ultrasound is used in combination with microbubbles and oncolytic virus to facilitate delivery to a particular region of the brain. Microbubbles are injected intravenously immediately before or during the procedure to deliver ultrasound to one or more central nervous system target(s). Opening of the BBB is then assessed by MRI evidence of extravasation of a contrast agent such as gadolinium (GAD) into the brain following intravenous injection. In another example, the ultrasound is delivered without MR guidance to a brain target using either frameless navigation and targeting of the ultrasound source to a planned surface area of the scalp, or the ultrasound source is inserted into the skull aimed at a planned trajectory to allow targeting of the ultrasound to a desired volume of brain tissue. In one example, the oncolytic viral agent may then be injected either simultaneously with the GAD contrast agent or up to 24 hours later. In one embodiment, the oncolytic virus is delivered intravenously.

In one embodiment, the agent that is administered systemically and transferred to the central nervous system target(s) with focused ultrasound mediated BBB disruption, is an oncolytic virus such as a herpes simplex virus. In one example, an oncolytic herpes simplex virus (HSV) with the thymidine kinase gene deleted is delivered to the grossly normal brain beyond the known limits of a tumor mass in order to target microscopically invasive tumor cells which cause recurrence. This virus is incapable of replicating in and destroying non-dividing cells, such as neurons and most quiescent glial cells in the brain, but this can replicate in and destroy dividing tumor cells which are actively synthesis nucleic acids for DNA replication necessary for viral DNA replication, thereby obviating the need for thymidine kinase. Given the large size of HSV compared with other viruses, such as AAV, as well as a very different surface protein content for the HSV envelope that could alter delivery across the BBB and spread of vectors compared with other, non-enveloped viruses, it was not obvious that this method would permit delivery of oncolytic viruses such as HSV or poliovirus. In another example, the oncolytic HSV also contains one or more additional genes or sequences, such as microRNAs or shRNAs, which can have additional anti-tumor cell activities by direct killing, facilitation of a drug or radiation therapy, or induction of anti-tumor immunity. In another example, the gene expressed from the oncolytic HSV encodes interleukin 12 (IL-12) to induce anti-tumor immunity.

In another example, the oncolytic virus is delivered in multiple sessions separated by at least 24 hours. Preferably, the oncolytic virus is delivered in multiple sessions separated by at least one week. Although one benefit of gene therapy agents over traditional chemotherapy is the more chronic nature of the ongoing therapy after delivery, it is still possible that tumor cells in different stages of cell division at any given session may not be equally susceptible to transduction or cell killing. Furthermore, most oncolytic viruses do not remain in the target tissue for more than a few days or weeks at most, as compared with other non-oncolytic agents such as AAV which can last for years or more. Therefore, any cells that escape killing in the first few days after a single oncolytic virus treatment may remain alive and capable of causing recurrence. However, it is also known that immune reactions to gene therapy agents can limit further transduction and efficacy, and oncolytic viruses that express immunomodulators, such as IL-12, might provoke further anti-viral immunity that could limit subsequent therapeutic sessions even while attempting to induce anti-tumor immunity. In this example, an oncolytic HSV expressing IL-12 is given systemically and delivered to an area of normal brain with the potential to target invasive tumor cells. One day after viral delivery, when the BBB should have been restored, gene expression from the HSV is observed robustly in the brain tissue targeted with ultrasound, roughly matching the ares of BBB disruption noted with GAD enhancement on MRI during the procedure. One week after HSV delivery, expression is almost gone, reflective of the relatively short-term nature of these agents. One day following a second ultrasound-mediated delivery of HSV-IL12 given one week after a first delivery of the same vector, robust expression is again observed in the brain tissue within the ultrasound field, demonstrating good repeat transduction that was not demonstrably limited due to exposure to the same agent at the same dose both systemically and in the same brain region one week earlier. Continued examination of animals treated in this fashion with two sessions of HSV-IL12 one week apart for several months after the second treatment showed no evidence of systemic illness, no loss of weight, no neurological deficits and no other evidence of significant brain or systemic injury from the multiple treatments.

In another embodiment, the oncolytic virus is delivered to brain tissue around a resection cavity following surgical removal or ablation of a tumor mass. Although many tumors are amenable to surgery, invasive brain tumors usually recur due to survival of cells which have invaded otherwise normal tissue outside of the main tumor mass. Since a main goal of tumor surgery is to resect a gross tumor mass without disturbing viable brain, these microscopic invasive cells usually evade the resection field and form the source of future recurrence. The presence of a resection cavity can limit the ability to deliver adjuvant therapies, such as oncolytic viruses, since the cavity results in a low resistance point for egress of viral agents following direct infusion, thereby limiting delivery to surrounding brain tissue. The presence of fluid rather than tissue within a resection cavity has the potential to also disrupt the dynamics of ultrasound transmission which could adversely influence the efficiency of blood-brain barrier disruption and therapeutic delivery to surrounding normal brain. In this embodiment, MRgFUS is used to effectively disrupt the BBB of brain tissue surrounding a resection cavity, resulting in efficient delivery of oncolytic herpes virus to the surrounding normal brain.

### Exemplary Oncolytic Viral Vectors

### Adenoviral Vectors

Adenovirus cannot integrate into the host genome and therefore mediates transient, rather than stable, gene expression. Adenoviral vectors are relatively easy to make and can be purified and concentrated up to 10¹² to 10¹³ viral particles/ml. In adenoviral vectors, the regions of at least the early adenoviral genes are usually replaced with an expression cassette containing the gene(s) of interest.

vectors, although all coding viral regions may be deleted so long as a packaging signal remains, thereby allowing for up to 36 kb of non-adenoviral sequences.

### Herpes Simplex Virus Vectors

Herpes simplex virus (HSV) has about a 152-kb genome and is a naturally neurotropic human virus, can maintain their presence in sensory neurons for long periods after infection. Two types of HSV vector systems are available: recombinant virus and amplicons. Recombinant virus can be divided into replication-competent attenuated vectors and replication-defective vectors. Replication-competent attenuated vectors contain essential genes for in vivo replication and retain the ability to multiply in actively dividing cells. Replication-defective vectors, on the other hand, are created by deleting immediate early (IE) genes that are required for replication, including the viral thymidine kinase (tk) gene. Nonessential viral genes in recombinant HSV vectors can be replaced by transgenes of interest at different sites in the viral genome, thereby allowing for up to 40 kb of transgenic DNA.

Amplicon vectors contain plasmid DNAs with only minimal HSV replication (ori) and packaging (pac) DNA sequences in the viral vector genome. Amplicons require HSV helper functions.

The methods described herein may be employed to prevent, inhibit or treat one or more brain cancers including but not limited to acoustic neuroma, astrocytoma, glioblastoma (GBM), chordoma, CNS lymphoma, craniopharyngioma, medulloblastoma, meningioma, metastatic brain tumors, oligodendroglioma, pituitary tumors, primitive neuroectodermal tumors (PNET), schwannoma, brain stem clioma, craniopharyngioma, ependymoma, juvenile pilocytic astrocytoma (JPA), medulloblastoma, optic nerve glioma, pineal tumor, or rhabdoid tumor.

### Exemplary Microbubbles

Microbubbles are smaller than one hundredth of a millimeter in diameter, but larger than one micrometer. In one embodiment, the microbubbles have an average diameter of about 1 to 10 μm. In one embodiment, the microbubbles have an average diameter of about 2 to 8 μm. In one embodiment, the microbubbles have an average diameter of about 10 to 100 μm. In one embodiment, the microbubbles have an average diameter of about 10 to 20 μm. In one embodiment, the microbubbles have an average diameter of about 20 to 80 μm. They include a shell that encapsulates a material, e.g., a shell that is gas-filled, e.g. air or perfluorocarbon. The shell may be formed of a lipid or a protein. In one embodiment, microbubbles are formed of a human serum protein such as serum albumin which encapsulates a gas such as perfluoropropane.

### Exemplary Conditions

In one embodiment for oncolytic viruses, an acoustic pressure amplitude of about 1 to about 3, e.g., about 2. MPa is employed. In one embodiment, an in situ acoustic pressure of about 0.5 to about 2.0, e.g., 1.02, MPa is attained. In one embodiment, a burst length of about 5 milliseconds to about 20 milliseconds (msec), e.g., 10 msec, is employed. In one embodiment, a pulse-repetition Frequency (PRF) of about 0.5 to about 2.5 Hz, e.g., about 1 Hz, is employed. In one embodiment, the period is about 500 msec to about 1500 msec, e.g., about 1000 msec. In one embodiment, the total sonication time is about 30 seconds to about 240 seconds, e.g., about 120 seconds.

The invention will be further described by the following non-limiting example.

### Example 1

### Materials and Methods

### Animal Preparation/Experimental Design

Four rats may be used to assess the efficiency and safety of unilateral MRgFUS-mediated HSV.GFP or HSVdelta tk delivery to the striatum at three weeks post sonication. Another group of fifteen rats may undergo a similar procedure but were sacrificed at different time points: 2 weeks (n=3), 2 months (n=4), 6 months (n=6) and 16 months (n=2). The brains and several organs (liver, lung, and heart) may then be harvested and processed for histological analysis.

### MRI-Guided Focused Ultrasound and Oncolvtic Viral Vector Delivery

Animals may be anesthetized using a Ketamine (90 mg/kg) and Xylazine (4 mg/kg) cocktail. A 22 g IV catheter (BD InsyteAutoguard) may be inserted into the lateral tail vein for substance administration during experiments. After scalp shaving, animals may be secured in a supine position on the FUS system and the head may be coupled with the degassed water tank holding the transducer. The spherically-focused transducer (7 cm diameter, f #=0.8) may be driven by a computer-controlled function generator (33220A Agilent Function/Arbitrary 20 MHz waveform generator; Agilent Technologies, CA) and a 43 db RF power amplifier (FUS Instruments, Inc).

Before sonication, an MRI may be performed with a 3.0T GE scanner, using a 4×7 cm RF surface coil. T2-weighted axial images, 10 slices, perpendicular to the direction of the ultrasound beam propagation, were acquired before sonication to calculate the coordinates of the target. The transducer may then be moved to the desired position using a motorized three-axis positioning system (FUS Instruments, Inc). The striatum may be sonicated in four points, 1.5 mm apart. Assuming a 49% loss of ultrasound power due to attenuation through the rat skull (Treat et al., 2007), an estimated in situ rarefactional pressure of 0.97 MPa may be applied at the sonication points, with a 1 Hz pulse repetition frequency, 10 ms burst length, and 200 s total sonication time. A cocktail of oncolytic virus (e.g., HSV) and Optison microspheres (Perflutren Protein-Type A microspheres, mean size 3-4.5 μm, 0.4×10⁸-0.64×10⁸/kg, GE Healthcare Life Sciences) may be administered simultaneously with sonication through the tail vein catheter, followed by MRI contrast agent Magnevist (gadopentetate dimeglumine, Gd-DTPA. 0.4 ml/kg; Bayer. Germany). TI-weighted images, 7 slices, may be collected at the conclusion of sonication to monitor the degree of the BBB opening based upon contrast extravasation. The slice thickness may be 0.8 mm, with a spacing of 0.2 mm.

After a tumor is resected from the brain, a fluid-filled cavity remains. After resection of an animal brain, tissue was targeted surrounding the cavity with focused ultrasound (where malignant cells would invade and where therapy could prevent recurrence). It was found that the blood brain barrier was effectively disrupted in these animals in the tissue immediately surrounding the cavity. Thus, focus ultrasound may be employed to target a tumor cavity to deliver agents including but not limited to drugs, antibodies, viruses or other vectors. In one embodiment, the virus may be an oncolytic virus or another virus expressing on or more anti-tumor genes.

### REFERENCES

Abbott et al., Neurobiol. Dis., 32:13 (2010).
Alonso et al., Mol. Ther. Nucleic Acids. 2:e73 (2013)
Bell et al., Hum. Gene Ther., 22:985 (2011).
Breous et al., Gastroenterology, 141:348-357, 357 e341-343, (2011).
Carty et al., J. Neurosci. Methods, 194:144 (2010).
Elias et al., N. Engl. J. Med., 369:640 (2013)
Elias et al., N. Engl. J. Med., 375:730 (2016).
Feigin et al., Proc. Natl, Acad. Sci. USA. 104:19559 (2007).
Fiandaca et al., Neuroimage. 42:T27 (2009).
Foley et al., J. Control Release, 196:71 (2014).
Fu et al., Gene Ther., 14:1065 (2007).
Fu et al., Mol. Ther., 5:911 (2003).
Hsu et al., PLoS One, 8:e57682 (2013).
Huang et al., Exp. Neurol., 233:350 (2012).
Hynynen et al., Radiology. 220:640 (2001).
Hynynen, Expert Opin. Drug Deliv., 4:27 (2007).
Jeanmonod et al., Neurosurg Focus, 32:E1 (2012).
Jordao et al., Exp. Neurol., 248:16 (2013).
Kaplitt et al., Lancet, 369:2097 (2007).
Kaplitt et al., Nat. Genet., 8:148 (1994).
Kovacs et al. Proc. Natl. Acad. Sci. USA, 114:E75 (2017).
LeWitt et al., Lancet Neurol., 10:309 (2011).
Manno et al., Nat. Med., 12:342 (2006).
Marks et al., Lancet Neurol., 7:400 (2008).
Marks et al., Lancet Neurol., 2:1164 (2010).
Mastakov et al., Mol. Ther., 2:225 (2001).
McCaffrey & Davis. J. Investig. Med., 60:1131 (2012).
McCarty et al., Gene Ther., 16:1340 (2009).
McDannold et al., Ultrasound Med. Biol., 31:1527 (2005).
Mingozzi & High, Curr. Gene Ther., 11:321 (2011).
Morgenstern et al., Methods Mol. Biol., 793:443 (2011).
Nance et al., J. Control Release, 189:123 (2014).
Neuwelt & Dahlborg, J. Neurooncol., 4:195 (1987).
Neuwelt et al., Ann. Intern. Med., 94:449 (1981).
Neuwelt et al., J. Clin. Invest., 64:684 (1979).
Neuwelt et al., J. Clin. Oncol., 9:1580 (1991).
Neuwelt et al., Neurosurgery, 12:662 (1983).
Neuwelt et al., Neurosurgery, 15:362 (1984).
Neuwelt et al., Neurosurgery, 17:419 (1985).
Rafii et al., Alzheimers Dement., 10:571 (2014).
Salegio et al., Adv. Drug Deliv. Rev., 64:598 (2012).
Schuster et al., Front Neuroanat., 8:42 (2014).
Thevenot et al., Hum. Gene Ther., 23:1144 (2012).
Treat et al., Int. J. Cancer, 121:901 (2007).
Treat et al., Ultrasound Med. Biol., 38:1716 (2012).
Tuszynski et al., JAMA Neurol., 72:1139 (2015).
Tuszynski et al., Nat. Med., 11:551 (2005).
Wang et al., Gene Ther., 12:1453 (2005).
Wang et al., Gene Ther., 22:104 (2015).
Wang et al., Sci. Rep., 7:39955 (2017).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method of delivering an oncolytic virus to one or more regions of a brain or spinal cord of a mammal having metastatic tumor cells within nervous system tissue, comprising: administering a first amount of an oncolytic virus to the mammal; and applying to one or more regions of the brain or spinal cord of the mammal suspected of having metastatic tumor cells focused ultrasound in an amount that provides for delivery of the oncolytic virus to the tumor cells.

2. The method of claim 1 wherein the oncolytic virus comprises a herpes simplex virus, poliovirus, reovirus or adenovirus.

3. The method of claim 1 further comprising subsequently administering a second amount of the oncolytic virus.

4. The method of claim 3 wherein the amount is administered at least one week after the first amount.

5. The method of claim 1 wherein the oncolytic virus is delivered to brain or spinal cord tissue surrounding a resection cavity.

6. The method of claim 1 wherein the oncolytic virus expresses a transgene capable of stimulating the immune system to attack tumor cells.

7. The method of claim 1 further comprising administering an amount of a population of microbubbles.

8. The method of claim 7 wherein the microbubbles comprise a mammalian serum protein.

9-10. (canceled)

11. The method of claim 7 wherein the microbubbles and the oncolytic virus are concurrently administered or a composition comprising the microbubbles and the oncolytic virus is administered.

12. (canceled)

13. The method of claim 7 wherein the focused ultrasound is applied after the microbubbles and the oncolytic virus are administered or wherein the focused ultrasound is applied concurrently with the administration of the microbubbles and the oncolvtic virus.

14-16. (canceled)

17. The method of claim 1 wherein the oncolytic virus comprises adenovirus or herpes simplex virus.

18. The method of claim 1 wherein the focused ultrasound is applied to the striatum, hippocampus, or basal forebrain.

19. A non-invasive method to deliver an anti-cancer agent to a resected portion of a brain or spinal cord of a mammal, comprising: administering to a mammal in need thereof, an amount of an oncolytic virus; and applying to one or more regions at or near the resected portion of the brain or spinal cord of the mammal focused ultrasound in an amount that provides for delivery of the oncolytic virus.

20. The method of claim 19 wherein the oncolytic virus comprises a herpes simplex virus, poliovirus, reovirus or adenovirus.

21. The method of claim 19 further comprising subsequently administering a second amount of the oncolytic virus.

22-23. (canceled)

24. The method of claim 19 further comprising administering an amount of a population of microbubbles.

25. The method of claim 24 wherein the microbubbles comprise a mammalian serum protein.

26. The method of claim 19 wherein the mammal is a human.

27. (canceled)

28. The method of claim 24 wherein the microbubbles and the oncolytic virus are concurrently administered or a composition comprising the microbubbles and the oncolvtic virus is administered.

29-30. (canceled)

31. The method of claim 24 wherein the focused ultrasound is applied concurrently with the administration of the microbubbles and the oncolytic virus.

32-35. (canceled)
